SEQUENCE LISTING (2 sequences)

SEQ ID NO: 1 - DNA, 84 bp, Artificial
  Oligonucleotide DR8F
ccggccatggcgaaaaacctgcgtcgcattatccgcaaaggcatccatatcattaaaaaatatggctagatggctctagacggc

SEQ ID NO: 2 - DNA, 18 bp, Artificial
  Oligonucleotide DRO
gccgtctagagccatcta

(19) United States
(12) Patent Application Publication
(10) Pub. No.: US 2009/0239757 A1
(43) Pub. Date: Sep. 24, 2009

(54) Control Sequences Responding to AMP and Uses Thereof

(75) Inventors: Jesper Duus Nielsen, Copenhagen (DK); Allan Kent Nielsen, Soeborg (DK); Bjarke Christensen, Lyngby (DK); Poul Erik Pedersen, Soeborg (DK); Hans-Henrik Kristensen Hoegenhaug, Holte (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/722,249

(22) PCT Filed: Dec. 20, 2005
(86) PCT No.: PCT/DK05/00807

§ 371 Date: Jun. 20, 2007

Related U.S. Application Data
(60) Provisional application No. 60/641,563, filed on Jan. 5, 2005.

(30) Foreign Application Priority Data
Dec. 23, 2004 (DK) PA 2004 02001

(51) Int. Cl. C40B 30/04; C12Q 1/68; C12N 1/21; C40B 30/06

(52) U.S. Cl. 506/9; 435/6; 435/252.3; 506/10

(57) ABSTRACT

The present invention relates to a method of identifying control sequences responding to antimicrobial peptides (AMPs), to host cells expressing an AMP and comprising a control sequence of the invention operable linked to a reporter, and to a method of screening for novel AMPs or AMP-variants having improved antimicrobial activity. Particularly the present invention relates to DNA control sequences, wherein the transcriptional profiles of said control sequences are regulated by the presence of an antimicrobial polypeptide expressed inside the host cell.

### FIELD OF THE INVENTION

The present invention relates to a method of identifying control sequences responding to antimicrobial peptides (AMPs), to host cells expressing an AMP and comprising a control sequence of the invention operable linked to a reporter, and to a method of screening for novel AMPs or AMP-variants having improved antimicrobial activity.

### BACKGROUND OF THE INVENTION

The rapid emergence of resistance to conventional antimicrobial agents is a growing problem in fighting bacterial and fungal infections. Various bioactive peptides are known to kill or inhibit the growth of target cells. Such bioactive peptides include antimicrobial enzymes, anti-tumor peptides and antimicrobial peptides (AMPs). In contrast to conventional antibiotics, such as penicillin, development of resistance to antimicrobial peptides is rare, and antimicrobial peptides are therefore an interesting alternative to marketed antibiotics. Antimicrobial peptides are found in a variety of plants and animals, where they are part of a first line of defense against infections, making them “natural peptide antibiotics”.

A method for identifying variants of known AMPs having improved antimicrobial activity have been described. WO 00/73433 describes a method for screening a pool of nucleotide sequences encoding an antimicrobial peptide operably linked to an inducible promoter and expressing the peptide in a host cell, wherein the peptides having improved antimicrobial activity are screened for by the effect of the inducer on the growth of the host cell.

Screening for growth is however not a very convenient and reliable parameter and therefore other parameters such as expression of a reporter gene is desirable.

Antimicrobial peptides are characterized by the potent and rapid killing of sensitive target cells. A hallmark of the peptides is that their minimal inhibitory concentration (MIC) is close to or equal to the minimal bactericidal concentration (MBC). As a result adding AMPs to the growth medium will rapidly kill the target cells. Hong et al. (2003, Antimicrobial agents and chemotherapy, 47: 1-6) describes examination of transcriptional profiles in E. coli in response to an antimicrobial peptide, which is added to the growth medium.

However, the effects of the AMPs on cell growth makes it difficult to obtain meaningful expression profiles by conventional methods, e.g. adding of sub-inhibitory concentrations of an AMP to a culture of cells as is often done with conventional antibiotics.

Improved screening methods for selecting novel AMPs or variants of such bioactive peptides having increased activity are thus desirable.

### SUMMARY OF THE INVENTION

To overcome the above mentioned problems associated with adding the AMP to the growth medium and the use of growth as the indicator for AMP efficiency, we have developed a new system where the AMP is produced by the cell itself. This has been shown to extend the growth inhibition phase and delay killing. The delay in killing of the organism in turn results in more meaningful expression profiles which are much easier to obtain experimentally. It is therefore possible from such expression profiles to identify suitable control sequences responsive to the presence of an AMP within the cell.

The present invention provides a method for identifying control sequences, such as promoters, which are responsive to the presence in the host cell of AMPs. These promoters are then subsequently used in a method for screening for novel AMPs or AMP-variants having improved antimicrobial properties by selecting for such improved properties by using suitable reporter genes operably linked to the said control sequences and utilizing the expression of the reporter in response to the AMP-variant as a selection/screening tool.

A first aspect of the invention relates to a method for identifying DNA control sequences, wherein the transcriptional profiles of said control sequences are regulated by the presence of an antimicrobial polypeptide, comprising the steps:

a) providing a nucleotide sequence encoding the antimicrobial polypeptide, and expressing the said antimicrobial polypeptide in a host cell;
b) isolating mRNA from the host cell in step (a); and
c) obtaining a transcription profile by analyzing the mRNA from step (b).

A second aspect of the invention relates to a host cell comprising a nucleotide sequence encoding a novel antimicrobial polypeptide or an antimicrobial polypeptide variant and a control sequence identifiable by the above method, wherein the control sequence is operably linked to a reporter gene.

A third aspect of the invention relates to a method of screening for novel antimicrobial polypeptides or antimicrobial polypeptide variants, comprising the steps of:

a) expressing a library of potential antimicrobial polypeptides or variants of antimicrobial polypeptides in a host cell according to the invention;
b) selecting cells on the basis of the reporter gene expression level;

A fourth aspect of the invention relates to a use of a control sequence obtainable by the method of the invention for screening for novel antimicrobial polypeptides and antimicrobial polypeptide variants.

### DETAILED DESCRIPTION OF THE INVENTION

The present invention take advantage of the surprising finding that promoter sequences identified by expression profiling by array analysis of mRNA isolated from bacterial cell grown in the presence of antimicrobial compounds are different from the promoter sequences identified according to the present invention. According to the present invention the antimicrobial polypeptide is expressed inside the host cell by controlled expression and at sub-lethal level. This will reduce the growth rate significantly but at the same time allow the cells to adapt their transcriptional profile to the new growth conditions.

By one of the methods according to the present invention promoter sequences responding to antimicrobial compounds are identified by expressing the antimicrobial compound, i.e. an antimicrobial peptide (AMP), within the bacterial cell and subsequently isolate mRNA from the bacterial cell and analyze the expression profile on micro arrays and compare these data with expression profiles of mRNA from bacteria grown without expressing the AMP.

A special advantage of the invention is the prolongation of the host response upon expression of an antimicrobial polypeptide as compared to external addition. When added to a culture of microorganisms, AMPs often exert their antimicrobial action within a few minutes killing the target organism. This makes it very difficult to obtain meaningful expression profiles using conventional methods described in the art. In the present invention, the exposure time can be extended to hours making it possible to obtain well-correlated and reproducible expression profiles.

By the present invention is provided a method for identifying DNA control sequences, wherein the transcriptional profiles of said control sequences are regulated by the presence of an antimicrobial polypeptide, comprising the steps:

a) providing a nucleotide sequence encoding the antimicrobial polypeptide, and expressing the said antimicrobial polypeptide in a host cell;
b) isolating mRNA from the host cell in step (a); and
c) obtaining a transcription profile by analyzing the mRNA from step (b).

“Transcriptional profiles” are in the present context corresponding to “expression profiles”. Such profiles provide a condition-specific and time-specific genome-scale snapshot of transcriptional activity, a profile of the response of the cell to its particular state and/or the conditions of its environment. The clusters of genes revealed by the collective array data can be thought of as an “expression profile”.

The term “control sequence” is defined herein to include all components which are necessary or advantageous for expression of the coding sequence of the nucleic acid sequence under control of the said sequence. Such control sequences include, but are not limited to, a promoter, a leader, a propeptide sequence, a signal sequence, and a transcription terminator. At a minimum, the control sequences include a promoter and associated and relevant regulatory sequences.

The control sequence may be an appropriate promoter sequence, a nucleic acid sequence which is recognized by a host cell for expression of the nucleic acid sequence. The promoter sequence contains transcription and translation control sequences which mediate the expression of the polypeptide.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

“Operably linked”, when referring to DNA segments, indicates that the segments are arranged so that they function in concert for their intended purposes, e.g. transcription initiates in the promoter and proceeds through the coding segment to the terminator.

A coding sequence is “under the control” of transcriptional and translational control sequences in a cell when RNA polymerase transcribes the coding sequence into mRNA, which is then translated into the protein encoded by the coding sequence.

The term “antimicrobial polypeptide” is defined herein to mean a polypeptide inhibiting or killing target microorganisms. The antimicrobial peptide (AMP) may be, e.g., a membrane-active antimicrobial peptide, or an antimicrobial peptide affecting/interacting with intracellular targets, e.g. binding to cellular DNA, RNA, enzymes or other essential cellular components. The AMP is generally a relatively short peptide, consisting of less than 200 amino acid residues, such as 5-15 amino acids, particularly 10-100 amino acids, more particularly 10-80 amino acids, typically 15-60 amino acid residues. The antimicrobial peptide has bactericidal and/or fungicidal effect, and it may also have antiviral or antitumour effects. It generally has low cytotoxicity against normal mammalian cells.

Membrane-active AMPs are often highly cationic and hydrophobic. It typically contains several arginine and lysine residues, and it may not contain a single glutamate or aspartate. It usually contains a large proportion of hydrophobic residues. The membrane-active peptide generally has an amphiphilic structure, with one surface being highly positive and the other hydrophobic.

The bioactive peptide and the encoding nucleotide sequence may be derived from plants, invertebrates, insects, amphibians and mammals, or from microorganisms such as bacteria and fungi.

The antimicrobial peptide may act on cell membranes of target microorganisms, e.g. through nonspecific binding to the membrane, usually in a membrane-parallel orientation, interacting only with one face of the bilayer.

The antimicrobial peptide typically has a structure belonging to one of five major classes: alpha-helical peptides, cystine-rich peptides (defensin-like), beta-sheet peptides, peptides with an unusual composition of regular amino acids, and peptides containing uncommon modified amino acids. Examples of alpha-helical peptides are Novispirins, Ovispirins, Magainin 1 and 2; Cecropin A, B and P1; CAP18; Andropin; Clavanin A or AK; Styelin D and C; and Buforin II. Examples of cystine-rich peptides are α-Defensin HNP-1 (human neutrophil peptide) HNP-2 and HNP-3; β-Defensin-12, Drosomycin, γ1-purothionin, Insect defensin A and Plectasin. Examples of β-sheet peptides are Lactoferricin B, Tachyplesin I, and Protegrin PG1-5. Examples of peptides with an unusual composition are Indolicidin; PR-39; Bactenicin Bac5 and Bac7; and Histatin 5. Examples of peptides with unusual amino acids are Nisin, Gramicidin A, and Alamethicin. Another example is the antifungal peptide (AFP) from Aspergillus giganteus.

Isolation of mRNA or total RNA is well known in the art and several commercial kits are available, e.g. High Pure RNA Isolation kit (Roche, cat #1 828 665).

The isolated mRNA is analyzed by e.g. micro arrays as explained below.

### DNA-Arrays

DNA microarrays are devices that provide a surface which has affixed sequences corresponding to some or all of the available open reading frames (ORFs) of a sequenced and annotated genome. Microarrays permit parallel recognition of sample sequences through hybridization of complementary base pairs to sequences on the array surface.

Gene expression profiling with cDNA microarrays has been used to obtain information on expression patterns, or profiles, of genes that are expressed in conjunction in response to or under various biological conditions. Schena et al. developed a high capacity system to monitor the expression of many genes in parallel utilizing microarrays (Quantitative monitoring of gene expression patterns with a complementary DNA microarray. Science (1996) 270, 467-470). Microarrays are now commonly prepared by a method of high speed robotic printing of cDNAs on glass. Such microarrays can provide a measure of the quantitative expression of the corresponding genes. In one such technique, differential expression measurements of genes are made by means of simultaneous, two color fluorescence hybridization. Other methods that may be used for differential expression measurement are, for example, oligochip, SAGE, differential display and its variants and subtractive hybridization.

By identifying and/or isolating genes whose expression differs between two cell or tissue types, or between cells exposed to different external conditions or stresses, such as physical extremes or an altered chemical environment, the underlying cellular processes and responses to environmental conditions are better understood. Furthermore, with arrays, a large segment of the genomic DNA can be measured simultaneously for such responses, with a profile of not just the presence, but the transcript abundance of each ORF simultaneously viewed for a given condition. This provides a condition-specific and time-specific genome-scale snapshot of transcriptional activity, a profile of the response of the cell to its particular state and/or the conditions of its environment.

The clusters of genes revealed by the collective array data can be thought of as an “expression profile”. Comparisons of such expression profiles can be used to highlight the regulatory networks and/or the metabolic and biosynthetic pathways that are active during the assayed conditions. While membership in a cluster implies co-regulation it also suggests that the products of the clustered genes carry out some common metabolic, biosynthetic or pathogenic function (Eisen et al. (1998) Cluster analysis and display of genome-wide expression patterns. Proc. Natl. Acad. Sci. (USA) 95, 14863-14868; Ferea, T. L., and Brown, P. O. (1999) Observing the living genome. Curr. Opin. Gen. Devel. 9, 715-722).

“Substrate” refers to a rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Array”, or “microarray” refers to an ordered arrangement of hybridizable array elements on a substrate. The array elements are arranged so that there are preferably at least ten or more different array elements. In alternative embodiments, at least 100 array elements, even more preferably at least 1000 array elements, and most preferably 10,000. The hybridization signal from each of the array elements is individually distinguishable. In a preferred embodiment, the array elements comprise nucleic acid molecules.

“Nucleic acid molecule” refers to a nucleic acid, oligonucleotide, nucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA). “Oligonucleotide” is substantially equivalent to the terms, primer, oligomer, element, target, and probe and is preferably single stranded.

“Up-regulated” refers to an increase in the level of reporter gene expression compared with the level of expression in an untreated sample.

“Down-regulated” refers to decrease in the level of reporter gene expression compared with the level of expression in an untreated sample.

Up- or down-regulation according to the invention is in one embodiment at least 2.5 fold, particularly at least 3 fold, more particularly at least 4 fold, more particularly at least 5 fold, more particularly at least 10 fold.

“Hybridization complex” refers to a complex between two nucleic acid molecules by virtue of the formation of hydrogen bonds between purines and pyrimidines.

The complete genome has been sequenced for a number of species of bacteria. The Comprehensive Microbial Resource (CMR) is a government funded initiative to encourage the sequencing of bacterial genomes, and to make the sequence information available to the community of researchers. The complete genomic sequences are made available through various publications, and the entire collection is maintained by The Institute for Genomic Research (TIGR). The CMR is fully described in: J. D. Peterson, L. A. Umayam, T. M. Dickinson, E. K. Hickey and O. White. The Comprehensive Microbial Resource. Nucleic Acids Research, 29:1 (2001), 123-125.

An array is a linear or two-dimensional arrangement of preferably discrete elements of nucleic acid sequences, each having a finite area, formed on the surface of a solid support.

A “microarray” will typically have elements with a density of at least about 100/cm², and preferably at least about 1000/cm². The elements in a microarray have typical dimensions, e.g., diameters, in the range of between about 10 to about 250 μm, preferably in the range of between about 10 to about 200 μm, more preferably in the range of between about 20 to about 150 μm, even more preferably in the range of between about 20 to about 100 μm, most preferably in the range of between about 50 to about 100 μm, and even most preferably in the range of between about 80 to about 100 μm, and are separated from other elements in the microarray by about the same distance.

Methods and instruments for forming microarrays on the surface of a solid support are well known in the art. See, for example, U.S. Pat. No. 5,807,522; U.S. Pat. No. 5,700,637; and U.S. Pat. No. 5,770,151. The instrument may be an automated device such as described in U.S. Pat. No. 5,807,522. DNA glass spotted microarrays have also been used for bacterial expression studies (Schoolnik, et al. (2001) Whole genome DNA microarray expression analysis of biofilm development by Vibrio cholerae O1 El Tor. Methods Enzymol. 336, 3-18; Wilson, et al. (2001) Functional genomics of Mycobacterium tuberculosis using DNA microarrays. Methods Mol. Med. 54, 335-358).

High density oligonucleotide arrays, manufactured by Affymetrix, Inc., Santa Clara, Calif., consist of 15 to 20 different 25-base oligonucleotides for each ORF of a sequenced genome; also represented in the same manner are intergenic regions greater than 200 bps (Lipshutz, R. J., Fodor, S. P., Gingeras, T. R. et al. (1999) High density synthetic oligonucleotide arrays. Nat. Genet. 21 (1 Suppl), 20-24; Lockhart, D. J., Byrne, M. C., Follettie, M. T. et al. (1996) Expression monitoring by hybridization to high-density oligonucleotide arrays. Nat Biotechnol. 14, 1675-1649; Harrington, C. A., Rosenow, C., and Retief, J. (2000) Monitoring gene expression using DNA microarrays. Curr. Opin. Microbiol. 3, 285-291). The selection of gene-specific oligonucleotides is based in part on sequence uniqueness in order to reduce cross-hybridization artifacts between paralogs, i.e., other genes in the genome that contain related sequences. Each oligonucleotide is paired with a so-called “mismatch” control oligonucleotide that differs from its “perfect match” partner by only one, centrally-located base. Comparison of the hybridization intensity of the perfect match and mismatch oligonucleotide provides a method for determining and subtracting background fluorescence.

Two-color hybridization employs two populations of cDNAs that have been differentially labeled with two different fluorochromes (ordinarily Cy3- and Cy5-dUTP) during a first-strand reverse transcription reaction using random hexamers as primers (Eisen, M. B., and Brown, P. O. (1999) DNA arrays for analysis of gene expression. Methods Enzymol. 303, 179-205). The resulting cDNAs are usually derived from RNA prepared from the same organism cultivated under, or exposed to, two contrasting conditions. Equal masses of the two differentially labeled populations of cDNAs are combined, applied to the array surface and allowed to hybridize to their corresponding ORF-specific targets. The array is then scanned and the intensity of each label for each ORF-specific spot is quantified. These values are compared, yielding ratios that serve as a measure of the relative degree of expression or repression of each ORF for the two tested conditions.

Membrane microarrays also contain robotically-printed PCR products corresponding to each of the annotated ORFs of a genome. However, unlike the DNA glass-spotted microarrays described above, membrane microarrays are produced by printing the double-strand amplicons onto positively-charged nylon membranes (Tao, H., Bausch, C., Richmond, C. et al. (1999) Functional genomics: expression analysis of Escherichia coli growing on minimal and rich media. J. Bacteriol. 181, 6425-6440). However, any type of substrate known in the art may be used in the methods of the present invention.

The delivery of a known amount of a selected nucleic acid on a specific position on the support surface is preferably performed with a dispensing device equipped with one or more tips for insuring reproducible deposition and location of the nucleic acids and for preparing multiple arrays. Any dispensing device known in the art may be used in the methods of the present invention. See, for example, U.S. Pat. No. 5,807,522. The diameter of each region is preferably between about 20-200 μm. The spacing between each region and its closest (non-diagonal) neighbor, measured from center-to-center, is preferably in the range of about 20-400 μm. Thus, for example, an array having a center-to-center spacing of about 250 μm contains about 40 regions/cm or 1,600 regions/cm². After formation of the array, the support is treated to evaporate the liquid of the droplet forming each region, to leave a desired array of dried, relatively flat regions. This drying may be done by heating or under vacuum. The DNA can also be UV-crosslinked to the polymer coating.

According to the method of the invention for selecting the appropriate control sequences responding to expression of an antimicrobial polypeptide within the host cell, cultures are grown under conditions providing expression of the antimicrobial polypeptide over a defined time course. Particularly the AMP is under the control of an inducible promoter in order to be able to control the level of AMP produced inside the host cell. The expression level of the AMP should not be so high as to kill the cell immediately. The skilled person will easily be able to determine appropriate levels of Induction which of cause will depend on the potency of the AMP. The choice of suitable inducible promoters depends on the host cell.

### Inducible Promoters

The expression vector used to express the polynucleotide sequence encoding an antimicrobial polypeptide according to the present invention should comprise an inducible promoter so that expression of said AMP may be controlled by a regulator. It is an advantage if the promoter allows tight regulation of the synthesis of the encoded AMP, i.e. that leakiness from the promoter is kept at a minimum. In addition, control of the transcription of the encoded AMP may particularly be significant as particularly short polypeptides are often inherently unstable and easily degraded in the cytoplasm of microorganisms. The inducible promoter may be regulated by more than one regulator. For example the promoter may be positively and negatively regulated, respectively, by two different compounds, e.g. in the presence of an inducer, expression from the promoter may be turned on; while in the absence of said inducer only very low levels of expression occur from the promoter. The uninduced (i.e. in the absence of the first inducer) levels may then be further repressed by the presence of a repressor. By varying the activity of the two regulators, protein expression levels may be manipulated to optimize expression of potentially toxic or essential genes.

One example of an inducible promoter and inducers is the Lac promoter as described in Taguchi S., Nakagawa K., Maeno M. and Momose H.; “In Vivo Monitoring System for Structure-Function Relationship Analysis of the antibacterial peptide Apidaecin”; Applied and Environmental Microbiology, 1994, pp. 3566-3572, which may be regulated by presence of the inducer lactose or by the synthetic non-digestible lactose derivative IPTG. Other examples include the trp promoters induced by tryptophan or gal promoters induced by galactose for E. coli, gall promoter for S. cerevisiae and AOX1 promoter for Pichia pastoris. It is an advantage to use an inducer which is not metabolized or digested in the host cell as this may keep the inducer concentration constant during the screening procedure.

Furthermore, it may be an advantage to select a promoter for which the corresponding inducer is able to permeate the cell membrane(s) to gain access to the promoter.

In a particular embodiment of the invention the promoter may be the pBAD promoter as used in the examples, vide infra, which is induced by the digestible inducer arabinose. If the pBAD promoter is used the host cells ability to digest arabinose may be eliminated by deleting the relevant genes from the host cell genome so as to achieve the above mentioned advantage of having a constant level of inducer.

The pBAD promoter is an example of a promoter which is regulated by two regulators as this is both positively and negatively regulated by AraC and cAMP-CRP. In the presence of arabinose, expression from the promoter is turned on, while in the absence of arabinose, only very low levels of expression occur from the promoter.

Uninduced levels may be even further repressed by culturing the cells in the presence of glucose. Glucose acts by lowering cAMP levels, which in turn decreases the binding of cAMP-CRP to the promoter region of pBAD. As cAMP levels are lowered, transcriptional activation is decreased. This is an advantage if the antimicrobial polypeptide of interest is extremely growth inhibitive or toxic to the host. In conclusion, by varying the activity of the two regulators, protein expression levels can be manipulated to optimize expression of potentially toxic or essential genes.

### Analysing Gene Expression Patterns/Profiles

The gene expression patterns derived from such treated biological samples can be compared with the gene expression patterns derived from untreated samples to identify and select nucleic acid molecules whose expression is either up-regulated or down-regulated due to the response to the AMP. One way of comparing gene expression profiles is by array analysis. Usually mRNA from the samples are converted to cDNA, however, the array analysis can also be performed using mRNA.

RNA can be isolated from the sample according to any of a number of methods well known to those of skill in the art. The sample nucleic acid molecules may be labeled with one or more labeling moieties to allow detection of hybridized arrayed/sample nucleic acid molecule complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. Preferred fluorescent markers include Cy3 and Cy5 fluorophores (Amersham Pharmacia Biotech, Piscataway N.J.).

After hybridization, the microarray is washed to remove nonhybridized nucleic acid molecules and complex formation between the hybridizable array elements and the nucleic acid molecules is detected. Methods for detecting complex formation are well known to those skilled in the art. In a preferred embodiment, the nucleic acid molecules are labeled with a fluorescent label and measurement of levels and patterns of fluorescence indicative of complex formation is accomplished by fluorescence microscopy, preferably confocal fluorescence microscopy.

In a differential hybridization experiment, nucleic acid molecules from two or more different biological samples are labeled with two or more different fluorescent labels with different emission wavelengths. Fluorescent signals are detected separately with different photomultipliers set to detect specific wavelengths. The relative abundances/expression levels of the nucleic acid molecules in two or more samples are obtained.

Typically, microarray fluorescence intensities can be normalized to take into account variations in hybridization intensities when more than one microarray is used under similar test conditions. In a preferred embodiment, individual arrayed-sample nucleic acid molecule complex hybridization intensities are normalized using the intensities derived from internal normalization controls contained on each microarray.

The preferred approach for comparing microarray data uses a similar procedure for extracting and developing the DNA samples for use with and printed on the microarray. PCR techniques can be used to isolate the whole, or nearly the whole, operon. This will mean that for some samples, a larger target is provided, and in some cases genes with marginal, or borderline changes in expression will be detected.

When unique oligos are chosen for formation of the microarray, each ORF will be represented by the same, or nearly the same, amount of DNA. One or more oligo may be used for each ORF, and the concentrations may vary. Preferred concentrations are in the range of 5 to about 20 μM.

In either case, it is important that the profile of genes expressed under certain conditions be developed using microarrays which have been prepared using same techniques, as between a control experiment and an treatment of the bacteria with an antimicrobial compound, or when evaluating the mode of action for a compound by comparing genes expressed in an experiment with a different compound.

The fluorescence or other data obtained from the scanned image may be analyzed using any of the commercially available image analysis software. The software preferably identifies array elements, subtracts backgrounds, deconvolutes multi-color images, flags or removes artifacts, verifies that controls have performed properly, and normalizes the signals (Chen et al., 1997, Journal of Biomedical Optics 2: 364-374).

Several computational methods have been described for the analysis and interpretation of microarray-based expression profiles including cluster analysis (Eisen et al., 1998, Proc. Nat. Acad. Sci. USA 95: 14863-14868), parametric ordering of genes (Spellman et al., 1998, Mol. Biol. Cell 9: 3273-3297), and supervised clustering methods based on representative hand-picked or computer-generated expression profiles (Chu et al., 1998. Science 282: 699-705).

Preferred methods for evaluating the results of the microarrays employ statistical analysis to determine the significance of the differences in expression levels. One such preferred system is the Significance Analysis of Microarrays (SAM) (Tusher, et al., (2001) Significance analysis of microarrays applied to the ionizing radiation response. Proc. Natl. Acad. Sci. (USA) 98, 5116-5121). Statistical analysis allows the determination of significantly altered expression of levels of about 50% or even less. The PAM (or predictive analysis for microarrays), represents another approach for analyzing the results of the microarrays (Tibshirani, et al, (2002) Diagnosis of multiple cancer types by shrunken centroids of gene expression. Proc. Natl. Acad. Sci. (USA) 99: 6567-6572).

Cluster algorithms may also be used to analyze microarray expression data. From the analysis of the expression profiles it is possible to identify co-regulated genes that perform common metabolic or biosynthetic functions. Hierarchical clustering has been employed in the analysis of microarray expression data in order to place genes into clusters based on sharing similar patterns of expression (Eisen, M. B., Spellman, P. T., Brown, P. O., et al., (1998) Cluster analysis and display of genome-wide expression patterns. Proc. Natl. Acad. Sci. (USA) 95, 14863-14868). This method yields a graphical display that resembles a kind of phylogenetic tree where the relatedness of the expression behavior of each gene to every other gene is depicted by branch lengths. The programs Cluster and TreeView, both written by Michael Eisen at Stanford University, are available at http://rana.stanford.edu/software/. Genespring is a commercial program available for such analysis.

Self-organizing maps (SOMs), a non-hierarchical method, have also been used to analyze microarray expression data (Tamayo, P. Slonim, D., Mesirov, J. et al. (1999) Interpreting patterns of gene expression with self-organizing maps: methods and application to hematopoietic differentiation. Proc. Natl. Acad. Sci. (USA) 96, 2907-2912). This method involves selecting a geometry of nodes, where the number of nodes defines the number of clusters. Then, the number of genes analyzed and the number of experimental conditions that were used to provide the expression values of these genes are subjected to an iterative process (20,000-50,000 iterations) that maps the nodes and data points into multidimensional gene expression space. After the identification of significantly regulated genes, the expression level of each gene is normalized across experiments. As a result, the expression profile of the genome is highlighted in a manner that is relatively independent of each gene's expression magnitude. Software for the “GENECLUSTER” SOM program for microarray expression analysis can be obtained from the Whitehead/MIT Center for Genome Research. SOMs can also be constructed using the GeneSpring software package.

In response to expression of a particular AMP, the array analysis will result in a number of possible control sequences/promoters which respond to the presence of the AMP within the cell. These promoters can either be up regulated or down regulated. In the examples a list of possible control sequences of the invention is shown, which are responding to expression of Novispirin. However, any suitable AMP can be used according to the invention, each of which might result in a different expression profile.

### Host Cells

The host cell according to the definition may be any cell susceptible to transformation or transfection with a nucleic acid construct. The host cell may be a unicellular microorganism, such as a prokaryote, or a non-unicellular microorganism, such as a eukaryote. Preferably for the method of identifying DNA control sequences responding to AMPs the host cells are those which have a significant portion of their genome sequenced.

Examples of unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus; or a Streptococci such as S. pneumonia, S. uberis, S. hyointestinalis, S. pyogenes or agalactiae; or a Staphylococci such as S. aureus, S. epidermidis, S. simulans, S. xylosus, S. camosus; or other Gram-positive bacteria such as Aerococcus viridans, Enterobacter cloacae, Enterococcus faecalis or Enterococcus hirae; or gram negative bacteria such as E. coli and Pseudomonas sp. such as P. aeruginosa; or other Gram-negative bacteria such as Acinetobacter baumanii, Bacteriodes fragilis, Bordetella bronchiseptica, Citrobacter freundii, Klebsiella pneumonia, Micrococcus luteus, Morganella morganii or Stenotrophomonas maltophila.

In a particular embodiment the bacterial cell is an ElectroMAX DH10B cell (GibcoBRL/Life technologies, UK) or of the genus E. coli, e.g. SJ2 E. coli of Diderichsen et al. (1990) or E. coli with the genotype: F⁻mcrA Δ(mrr-hsdRMS-mcrBC) Φ80lacZΔM15 ΔlacX74 deoR recA1 araD139 Δ(araA-leu)7697 galU galK rpsL endA1 nupG, also known as the commercially available TOP10 cells from Invitrogen. Other particular host cells may be strains of Bacillus, such as Bacillus subtilis or Bacillus sp. A particular useful eukaryotic cell is a yeast, e.g. S. cerevisae.

For some polypeptides it may be important for their activity that a disulfide bond is formed between two cysteine residues in the polypeptide. In some host cells, e.g. yeast, formation of disulfide bonds often takes place as a natural part of the expression of polypeptides. However, for other types of host cells it doesn't. Thus in a particular embodiment the redox state of the host cell may be such that it allows disulfide bond formation. This is particularly important if the activity of the antimicrobial polypeptide is affected by the presence of disulfide bond(s) in said polypeptide. The host cell may be such that it naturally allows disulfide bond formation or it may harbour one or more mutations that allow disulfide bond formation. For example the host cell may harbour a mutation in the thioredoxin reductase gene (trxB) and/or in the glutathione reductase gene (gor). In particular the host cell may be a K-12 derivative of E. coli harbouring mutations in both the thioredoxin reductase gene and the glutathione reductase gene, such as the commercially available E. coli origami cells from Novagen.

The host cell may also be a eukaryote, such as a mammalian, insect, plant, or fungal cell.

In a particular embodiment, the host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more particular embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

Examples of yeast host cell include Candida, Hansenula, Kluyveromyces, e.g. K. lactis, Pichia, Saccharomyces, e.g. S. carlsbergensis, S. cerevisiae, S. diastaticus, S. douglasii, S. kluyveri, S. norbensis or S. oviformis, Schizosaccharomyces, or Yarrowia cell, e.g. Y. lipolytica.

In another embodiment the fungal host cell may be a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligating aerobic. In contrast, vegetative growth by yeasts such as Saccharomyces cerevisiae is by budding of a unicellular thallus and carbon catabolism may be fermentative.

Examples of filamentous fungal host cell include a cell of Acremonium, Aspergillus, e.g. A. awamori, A. foetidus, A. japonicus, A. nidulans, A. niger or A. oryzae, Fusarium, e.g. F. bactridioides, F. cerealis, F. crookwellense, F. culmorum, F. graminearum, F. graminum, F. heterosporum, F. negundi, F. oxysporum, F. reticulatum, F. roseum, F. sambucinum, F. sarcochroum, F. sporotrichioides, F. sulphureum, F. torulosum, F. trichothecioides, or F. venenatum, Humicola, e.g. H. insolens, H. lanuginose, Mucor, e.g. M. miehei, Myceliophthora, e.g. M. thermophila, Neurospora, e.g. N. crassa, Penicillium, e.g. P. purpurogenum, Thielavia, e.g. T. terrestris, Tolypocladium, or Trichoderma, e.g. T. harzianum, T koningii, T. longibrachiatum, T. reesei, or T. viride.

Methods for cloning genes under control of inducible promoters are well known in the art. For a general reference to molecular cloning techniques see e.g. Sambrook, Fritsch and Maniatis, Molecular Cloning, A laboratory manual. Cold Spring Habor Laboratory Press, 1989). The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Identification of promoter sequences responding to a given AMP can subsequently be used to select a promoter, which e.g. responds to expression of the AMP by an increased promoter activity. Particularly the promoter activity may be up-regulated at least about 100%, more preferably about 200%, as measured by the relative signal of the detection method.

The promoter can then be used for controlling a reporter gene in a host cell which is transformed with a library of potential antimicrobial polypeptides or polypeptide variants of a given AMP, and preferably the AMP used for selecting the promoter. The variants are in a particular embodiment controlled by an inducible promoter. When the variant are grown under conditions providing induction of the AMP the promoter sequence selected by the method of the invention will respond and express the reporter gene. The expression of the reporter can thus be used for selecting variants of the AMP having desirable properties such as e.g. enhanced activity.

In a further aspect the invention therefore relates to a host cell comprising a nucleotide sequence encoding a novel antimicrobial polypeptide or an antimicrobial polypeptide variant and a control sequence identifiable by the method of the invention, wherein the control sequence is operably linked to a reporter gene.

The control sequence should preferably be induced by the presence or synthesis of the antimicrobial polypeptide in question. The control sequence can be fused to the reported gene by either transcriptional and/or translational fusions and propagated by chromosomal integration or extrachromosomal elements such as plasmids or phages or phagemides. The preference of one fusion method over the other is often regulated by the specific reporter system and/or promoter system.

Suitable reporter genes include but are not limited to beta-galactosidase, chloramphenicol acetyltransferase, luciferase, GFP, RFP, BFP, YFP as well as miscellaneous drug resistance markers. The specific reporters are chosen on the basis of the anticipated screening method eg. Fluorescence markers such as GFP, RFP, BFP and YFP will allow for high-throughput setup using fluorescence-assisted cell sorting (FACS).

Markers resulting in a chromogenic readout such as beta-galactosidase or chloramphenicol acetyltransferase would allow for screenings in liquid or solid growth media such as agar substrates or microtiter plates.

The invention is not limited to the use of one promoter or reporter system. A reported strain could contain two or more different promoter-reporter systems.

### Identification of Suitable Control Sequences Responding to Intracellular Expression of AMP.

In the examples below the method of the invention has been illustrated using novispirin as the antimicrobial peptide and E. coli as the host cell. To assess the sustained physiological response of E. coli to novispirin, an arabinose induction system was used to make E. coli produce novispirin at a sub-lethal rate. Induction of novispirin expression reduced the growth rate significantly, but the growth was not completely inhibited, thereby allowing the cells to adapt their transcriptional profile to the new growth conditions. Based on a confidence level of 90%, microarray analysis showed that 200 genes were up-regulated and 144 genes were down-regulated relative to a control strain that expressed a peptide without antimicrobial activity. The transcriptional response was highly coordinated, allowing the majority of the highly up-regulated genes to be assigned to categories according to their annotated physiological functions. These categories comprise particularly genes related to membrane dysfunction, heat shock, phage shock and antimicrobial peptide defense.

In a particular embodiment the control sequence comprises promoters chosen from the subgroups comprising promoters controlling genes involved in acid shock, osmotic shock, membrane biogenesis and repair, redox, phage shock, protein folding, AMP resistance, cell wall, nitrate assimilation, carbon metabolism or heat shock.

In another particular embodiment the promoters control genes involved in acid shock, osmotic shock or phage shock.

In a further particular embodiment the genes comprises Asr, gadB, gadC, KatE, OsmE, ProW and pspABCDE.

In a further aspect the invention relates to a method of screening for novel antimicrobial polypeptides and antimicrobial polypeptide variants, comprising the steps:

a) expressing a library of potential antimicrobial polypeptides or antimicrobial polypeptide variants in a host cell comprising a nucleotide sequence encoding a novel antimicrobial polypeptide or an antimicrobial polypeptide variant and a control sequence identifiable by the method of the invention, wherein the control sequence is operably linked to a reporter gene;
b) selecting cells on the basis of the reporter gene expression level;

The selected cells from step (b) can in a further step (c) be purified and the novel antimicrobial polypeptide or antimicrobial polypeptide variants can be isolated and identified the from the purified cells having up- or down-regulated reporter gene expression levels compared to an identical cell expressing the no antimicrobial polypeptide or parent antimicrobial polypeptide.

In some situations an increase or up-regulation in expression will be more suitable for selection, however, also a decrease or down-regulation could work as a basis for selection.

According to the present invention it could therefore also be envisioned that a promoter displaying a reduced expression in response to expression of a given AMP. Such a promoter could also be applied in the above method.

The selection of cells based on the reporter gene expression level can be performed in many ways dependent on the reporter gene of choice. One way, which is suitable, when the reporter gene is chosen among the fluorescent proteins like e.g. GFP, is sorting by FACS.

Another way could be selecting colonies by visual inspection on X-gal plates in case the reporter gene is lacZ.

### EXAMPLES

### Example 1

### Construction of a Plasmid Conditionally Expressing Novispirin in E. coli

A plasmid was constructed to investigate the transcriptional response of E. coli upon expressing Novispirin G10 in a Suicide Expression System (SES) setup. The SES takes advantage of conditionally expressing antimicrobial peptides in a sensitive host. The degree of inhibition in the host reflects the potency of the antimicrobial peptide (AMP) (WO200073433). In the current setup, the AMP is exported to the periplasmic space where it interacts with the inner and/or outer membrane of E. coli. Expressing the AMP, as opposed to adding purified peptide to a cell culture, was shown to prolong the inhibition and delay killing.

The AMP, Novispirin G10 which is a potent 18 amino acid antimicrobial peptide with activity against both Gram-positive and Gram-negative bacteria (U.S. Pat. No. 6,492,328) was used.
The control plasmid employed was pBAD/gill A (Invitrogen, USA).

The plasmid expressing Novispirin G10, pDRS5-Novispirin, was constructed by annealing the oligonucleotides DR8F (5′-ccg gcc atg gcg AAA MC CTG CGT CGC ATT ATC CGC MA GGC ATC CAT ATC ATT AAA AAA TAT GGC tag atg gct cta gac ggc-3′) and DRO (5′-GCC GTC TAG AGC CAT CTA-3′) and followed by filling out using 5 units (1 μL/100 μL) Taq Pwo polymerase, 0.25 mM deoxynucleotide triphosphate, 1 μM DR8F primer, 1 μM DRO primer in 100 μL standard PCR buffer. One cycle of a PCR program consisting of 2 min at 94° C., 3 min at 50° C. and 10 min at 72° C. was used.

The PCR product was digested with NcoI/XbaI under conditions described by the manufacturer (New England Biolabs, US) and the gene was inserted directionally into a pBAD gIII A vector (Invitrogen, US). The ligation mixture was then transformed into E. coli TOP10 cells (invitrogen, US) using the standard heat shock method with a 30 second heat shock at 42° C. followed by 1 hour at 37° C. in LB media with 100 mg/L ampicillin. Hereafter, the cells were grown overnight at 37° C. on LB plates containing 100 mg/L ampicillin. Colonies were picked, transferred to new LB plates, and the correct insertion of the gene was verified by DNA sequencing.

### Example 2

### Cultivating E. coli/pDRS5 Under Inducing Conditions

In order to obtain a transcriptional profile of E. coli expressing Novispirin G10, 2×10 mL sterile LB media (10 g/L Bactotryptone, 5 g/L Yeast extract, and 10 g/L NaCl) containing 100 mg/L ampicillin was inoculated with E. coli TOP10 harbouring pDRS5-novispirin and pBAD gIII A, respectively. These cultures were grown overnight at 37° C. at 180 rpm. The overnight cultures were diluted 1/100 into fresh sterile RM media (2% Casamino Acids, 0.2% glycerol, 1 mM MgCl₂, 0.1 mM thiamine, 6 g/L Na₂HPO₄, 3 g/L KH₂PO₄.2H₂O, 0.5 g/L NaCl, 0.1 g/L NH₄Cl at pH 7.4) supplemented with 100 mg/L ampicillin and 0.1% arabinose. 100 mL of each culture was grown at 37° C., 240 rpm for 4.5 hours before being harvested by poured into centrifugation tubes containing 10 mL pre-frozen milliQ water and immediate centrifugation at 4200 g for 15 minutes at 4° C. Immediately hereafter, the supernatant was decanted and the cell supernatant was frozen at −20° C. for storage until RNA preparation was initiated.

### Example 3

### RNA Isolation and Labelling

Total RNA was isolated from the two E. coli (expressing either Novispirin G10 or a control peptide) cultures by use of the High Pure RNA Isolation kit (Roche, cat #1 828 665) according to the manufacturers instructions. Residual DNA was removed on-column with RNase-free DNase. Labelled samples were prepared from 30 μg of total bacterial RNA. Fluorescent first strand cDNA was prepared by random primed reverse transcription (Superscript II; Life Technologies) by use of random hexamers for cDNA synthesis. 2 μl (20 μg) of random primer was mixed with 15 μl (30 μg) of total RNA, incubated for 5 min at 70° C., and cooled on ice. To each sample was added 5× first strand buffer (6 μl), 0.1 M DTT (3 μl), dNTP mix (5 mM dATP, dGTP and dTTP and 2 mM dCTP) (1 μl), RNaseOut (1 μl), Superscript II reverse transcriptase (2 μl) and Cy-dCTP (3 μl). Reverse transcription was carried out at 42° C. for 1 hr. RNA was removed by addition of 10 μl NaOH (1 M) and incubation at 65° C. for 10 min. The samples were neutralised by addition of 10 μl HCl (1 M). Cy3 and Cy5 cDNA samples were mixed and immediately purified with a GFX PCR DNA and Gel Band Purification Kit (Amersham Biosciences).

### Example 4

### Hybridizations, Scanning and Analysis

Slides were pre-hybridised at 42° C. for 45 min in a solution containing 4×SSC+0.5% SDS+1% BSA and dried by centrifugation. Hybridizations were in a volume of 25 μl under a supported cover slip at 42° C. for 16-18 h at high humidity. The hybridisation mixture was labelled cDNA in a formamide-based hybridisation buffer (MWG—supplied with the pan-arrays). Cover slip and supports were gently removed (2×SSC in a wash tray) and arrays were washed by immersion into 2×SSC+0.1% SDS for 5 min, 1×SSC+0.1% SDS for 5 min, 0.5×SSC for 5 min and 1× millipore water for 10 sec and dried by centrifugation. Slides were scanned on a GMS 418 scanner to detect Cy3 and Cy5 fluorescence. Data analysis was carried out using ImaGene 5.0 and GeneSight 3.5 from BioDiscovery (www.biodiscovery.com). Two biological replicate experiments with four replicate hybridisations in each experiment were carried out for generation of data.

Pan E. coli K12 Arrays from MWG Biotech were used in this study (lot No.: 020206). The 4239 E. coli K12 specific oligonucleotide probes present on this glass-based DNA chip represent the complete Escherichia coli (K12) genome

### Example 5

### Genes Significantly Up- or Down-Regulated Upon Expression of Novispirin G10

Gene expression data was analyzed using the GeneSight 4.1.5 software from BioDiscovery. Data was prepared using local background correction, removal of flagged spots (contaminated background or signal), floor value raising data to a minimum value of 20, calculation of ratio, log(2) transformation of ratios, print-tip lowess normalization, and finally combination of replicate spot.

TABLE 1

| Gene | ID | ratio |
|---|---|---|
| orf, hypothetical protein; | b1721 | 98.3 |
| probable third cytochrome oxidase, subunit II; | appB | 47.2 |
| transketolase 2 isozyme; | tktB | 26.5 |
| acid sensitivity protein, putative transporter; | xasA | 24.9 |
| acid shock protein; | asr | 19.2 |
| phage shock protein: activates phage shock-protein expression; | pspC | 15.8 |
| nitrite extrusion protein 2; | narU | 15.1 |
| aerotaxis sensor receptor, flavoprotein; | aer | 14.3 |
| protein induced by aluminum; | ais | 14.1 |
| phage shock protein; | pspD | 13.5 |
| possible regulator of murein genes; | bolA | 13.0 |
| orf, hypothetical protein; | yohC | 12.0 |
| orf, hypothetical protein; | yeaG | 12.0 |
| transaldolase A; | talA | 11.7 |
| orf, hypothetical protein; | crcA | 11.7 |
| activator of ntrL gene; | osmE | 11.7 |
| putative chaperone; | sfmC | 11.4 |
| cryptic nitrate reductase 2, beta subunit; | narY | 11.0 |
| orf, hypothetical protein; | yiaG | 10.1 |
| catalase; hydroperoxidase HPII(III); | katE | 9.9 |
| orf, hypothetical protein; | b3100 | 9.9 |
| glycoprotein/polysaccharide metabolism; | ybaY | 9.9 |
| putative transferase; | b1454 | 9.7 |
| orf, hypothetical protein; | elaB | 9.7 |
| phage shock protein; | pspE | 9.4 |
| orf, hypothetical protein; | ybhL | 9.3 |
| orf, hypothetical protein; | b1724 | 9.1 |
| putative glutaminase; | ybaS | 8.8 |
| putative resistance protein; | ylcD | 8.8 |
| glucosamine-6-phosphate deaminase; | nagB | 8.8 |
| orf, hypothetical protein; | b1825 | 8.7 |
| putative enzyme; | b2253 | 8.4 |
| putative ligase; | ybaP | 8.4 |
| orf, hypothetical protein; | yjbO | 8.3 |
| glutamate-1-semialdehyde aminotransferase (aminomutase); | hemL | 8.3 |
| transport permease protein of gamma-aminobutyrate; | gabP | 8.2 |
| orf, hypothetical protein; | b2097 | 8.1 |
| high-affinity transport system for glycine betaine and proline; | proW | 7.9 |
| orf, hypothetical protein; | ygaM | 7.9 |
| orf, hypothetical protein; | hdeB | 7.9 |
| orf, hypothetical protein; | hdeA | 7.7 |
| orf, hypothetical protein; | yohM | 7.7 |
| Mg2 transport ATPase, P-type 1; | mgtA | 7.5 |
| orf, hypothetical protein; | ycaC | 7.4 |
| osmotically inducible protein; | osmC | 7.2 |
| putative transport system permease protein; | yehZ | 7.1 |
| orf, hypothetical protein; | b1836 | 6.9 |
| hypothetical protein; | yeaQ | 6.9 |
| phage shock protein; | pspB | 6.9 |
| response regulator for chemotaxis; protein glutamate methyltransferase; | cheR | 6.8 |
| orf, hypothetical protein; | b1582 | 6.8 |
| phage shock protein, inner membrane protein; | pspA | 6.7 |
| orf, hypothetical protein; | yciR | 6.5 |
| 4-aminobutyrate aminotransferase activity; | gabT | 6.5 |
| probable regulator for maltose metabolism; | sfsA | 6.4 |
| hyperosmotically inducible periplasmic protein; | osmY | 6.4 |
| putative cytochrome; | yodB | 6.2 |
| putative enzyme; | yaiH | 6.1 |
| orf, hypothetical protein; | ybjG | 5.8 |
| NAD-linked malate dehydrogenase (malic enzyme); | sfcA | 5.8 |
| cytoplasmic alpha-amylase; | amyA | 5.8 |
| orf, hypothetical protein; | yqjD | 5.8 |
| putative transcriptional regulator LYSR-type; | ynfL | 5.7 |
| orf, hypothetical protein; | yggH | 5.7 |
| trehalose-6-phosphate phophatase, biosynthetic; | otsB | 5.6 |
| putative ATP-binding component of a transport system; | yehX | 5.6 |
| putative sugar transferase; | b2254 | 5.5 |
| orf, hypothetical protein; | yciE | 5.4 |
| flagellar biosynthesis; filament capping protein; enables filament assembly; | fliD | 5.4 |
| orf, hypothetical protein; | b1837 | 5.3 |
| putative enzyme; | ybjX | 5.3 |
| orf, hypothetical protein; | ycfH | 5.3 |
| orf, hypothetical protein; | ygiW | 5.2 |
| protein phosphatase 1 modulates phosphoproteins, signals protein misfolding; | pphA | 5.2 |
| 30S ribosomal subunit protein S22; | rpsV | 5.2 |
| orf, hypothetical protein; | ygeF | 5.2 |
| trehalose-6-phosphate synthase; | otsA | 5.2 |
| orf, hypothetical protein; | b2833 | 5.1 |
| orf, hypothetical protein; | yciI | 5.1 |
| orf, hypothetical protein; | ygaU | 5.1 |
| orf, hypothetical protein; | ymfM | 5.1 |
| orf, hypothetical protein; | b1355 | 5.0 |
| orf, hypothetical protein; | yccD | 4.9 |
| orf, hypothetical protein; | yfbM | 4.9 |
| outer membrane lipoprotein (lipocalin); | blc | 4.9 |
| cryptic nitrate reductase 2, alpha subunit; | narZ | 4.9 |
| orf, hypothetical protein; | b1450 | 4.8 |
| outer membrane protein X; | ompX | 4.7 |
| putative peptidase; | pepB | 4.7 |
| membrane channel protein component of Pn transporter; | phnE | 4.6 |
| orf, hypothetical protein; | b1826 | 4.6 |
| periplasmic maltose-binding protein; | malE | 4.6 |
| flagellar biosynthesis; | fliQ | 4.5 |
| orf, hypothetical protein; | yjdB | 4.5 |
| orf, hypothetical protein; | b1643 | 4.5 |
| induced in stationary phase, recognized by rpoS, affects cell division; | fic | 4.5 |
| putative sporulation protein; | ycgB | 4.5 |
| putative transport; | ybbY | 4.4 |
| global regulator, starvation conditions; | dps | 4.4 |
| orf, hypothetical protein; | yhcO | 4.4 |
| orf, hypothetical protein; | b1583 | 4.3 |
| orf, hypothetical protein; | hdeD | 4.3 |
| putative protease; htrA suppressor protein; | sohA | 4.3 |
| orf, hypothetical protein; | ygiA | 4.2 |
| orf, hypothetical protein; | ybgA | 4.2 |
| orotate phosphoribosyltransferase; | pyrE | 4.2 |
| putative transport system permease protein; | yehY | 4.2 |
| putative a membrane protein; | ybdS | 4.2 |
| phospho-beta-glucosidase; cryptic; | celF | 4.1 |
| dnaK suppressor protein; | dksA | 4.1 |
| orf, hypothetical protein; | yijC | 4.1 |
| orf, hypothetical protein; | yqhE | 4.0 |
| PTS system fructose-like IIB component 1; | frwB-r | 4.0 |
| tetrahydropteroyltriglutamate methyltransferase; | metE | 4.0 |
| periplasmic protein of mal regulon; | malM | 4.0 |
| orf, hypothetical protein; | b2256 | 4.0 |
| ATP-binding component of putrescine transport system; | potG | 4.0 |
| putative glycosidase; | yihQ | 3.9 |
| outer membrane channel; specific tolerance to colicin E1; | tolC | 3.9 |
| orf, hypothetical protein; | b3023 | 3.9 |
| ribosome modulation factor; | rmf-r | 3.9 |
| orf, hypothetical protein; | ymgE | 3.9 |
| restriction of DNA at 5-methylcytosine residues; at locus of e14 element; | mcrA | 3.9 |
| RNase T, degrades tRNA; | rnt | 3.9 |
| orf, hypothetical protein; | yqjE | 3.9 |
| orf, hypothetical protein; | yhiE | 3.9 |
| alcohol dehydrogenase; | adhP | 3.8 |
| orf, hypothetical protein; | b1953 | 3.8 |
| putative transport protein, cryptic, orf, joins former yjiZ and yjjL; | yjiZ | 3.8 |
| N-acetylglutamate synthase; amino acid acetyltransferase; | argA | 3.8 |
| putative receptor protein; | b2372 | 3.8 |
| orf, hypothetical protein; | ygaG | 3.8 |
| spermidine N1-acetyltransferase; | speG | 3.8 |
| thymidine phosphorylase; | deoA | 3.8 |
| periplasmic chaperone, required for type 1 fimbriae; | fimC | 3.8 |
| orf, hypothetical protein; | yigW_1 | 3.8 |
| sensor protein PhoQ; | phoQ | 3.7 |
| orf, hypothetical protein; | ygfQ | 3.7 |
| N-acetylglucosamine-6-phosphate deacetylase; | nagA | 3.7 |
| putative amino acid/amine transport protein; | b1691 | 3.7 |
| orf, hypothetical protein; | yedU | 3.7 |
| orf, hypothetical protein; | yqjF | 3.6 |
| sensor histidine protein kinase (RstA regulator); | rstB | 3.6 |
| putative alpha helical protein; | ybeL | 3.6 |
| outer membrane protein induced after carbon starvation; | slp | 3.6 |
| D-alanyl-D-alanine carboxypeptidase; penicillin-binding protein 6; | dacC | 3.6 |
| orf, hypothetical protein; | ybfA | 3.5 |
| D-mannonate oxidoreductase; | uxuB | 3.5 |
| orf, hypothetical protein; | b1627 | 3.5 |
| probable third cytochrome oxidase, subunit I; | appC | 3.5 |
| orf, hypothetical protein; | b2809 | 3.4 |
| cytosine deaminase; | codA | 3.4 |
| putative oxidoreductase; | yncB | 3.4 |
| phenylalanine-specific transport system; | pheP | 3.4 |
| orf, hypothetical protein; | yhfG | 3.4 |
| vitamin B12 transport; | btuE | 3.4 |
| glucokinase; | glk | 3.4 |
| sn-glycerol 3-phosphate transport system, integral membrane protein; | ugpA | 3.3 |
| putative 2-component transcriptional regulator; | uvrY | 3.3 |
| putative xanthine dehydrogenase (EC 1.1.1.20); | yagT | 3.3 |
| putative polysaccharide export protein; | wza | 3.3 |
| pyruvate oxidase; | poxB | 3.3 |
| probable enzyme; | b1408 | 3.3 |
| glyceraldehyde 3-phosphate dehydrogenase C, interrupted; | gapC_1 | 3.3 |
| homoserine kinase; | thrB | 3.3 |
| dihydrofolate reductase type 1; trimethoprim resistance; | folA | 3.2 |
| orf, hypothetical protein; | yqjC | 3.2 |
| heat shock protein, chaperone, member of Hsp70 protein family; | hscA | 3.2 |
| orf, hypothetical protein; | b1520 | 3.2 |
| orf, hypothetical protein; | yjcF | 3.1 |
| respiratory NADH dehydrogenase; | ndh | 3.1 |
| putative ATP-binding component of a transport system; | yrbF | 3.1 |
| membrane-bound ATP synthase, F1 sector, epsilon-subunit; | atpC | 3.1 |
| orf, hypothetical protein; | yjiG | 3.1 |
| orf, hypothetical protein; | yfcI | 3.1 |
| putative ARAC-type regulatory protein; | yhiW | 3.0 |
| lipoprotein precursor; | vacJ | 3.0 |
| putative arginine/ornithine antiporter; | b1605 | 3.0 |
| hypothetical protein; | yddM | 3.0 |
| orf, hypothetical protein; | b1354 | 3.0 |
| phosphopentomutase; | deoB | 3.0 |
| orf, hypothetical protein; | ycgY | 3.0 |
| putative transcriptional regulator LYSR-type; | b1422 | 3.0 |
| transcriptional represser for deo operon, tsx, nupG; | deoR | 2.9 |
| orf, hypothetical protein; | yaaI | 2.9 |
| putative proteoglycan; | b2879 | 2.9 |
| dihydroxynaphtoic acid synthetase; | menB | 2.9 |
| restriction of methylated adenine; | mrr | 2.9 |
| putative transferase; | yqjG | 2.9 |
| orf, hypothetical protein; | yhiF | 2.9 |
| putative oxidoreductase; | yohF | 2.8 |
| orf, hypothetical protein; | yccM | 2.8 |
| orf, hypothetical protein; | ycfR | 2.8 |
| putative enzyme; | b1394 | 2.8 |
| orf, hypothetical protein; | yeaH | 2.8 |
| putative transport protein; | yoaE | 2.8 |
| transcriptional regulatory protein; | phoP | 2.8 |
| curli production assembly/transport component, 2nd curli operon; | csgF | 2.8 |
| RNA polymerase, beta subunit; | rpoB-r | 2.8 |
| putative enzyme; | ysgA | 2.8 |
| orf, hypothetical protein; | ybhS | −2.6 |
| UDP-D-glucose: (galactosyl)lipopolysaccharide glucosyltransferase; | rfaJ | −2.6 |
| flagellar biosynthesis, cell-distal portion of basal-body rod; | flgG | −2.6 |
| orf, hypothetical protein; | yeaP | −2.6 |
| transport of long-chain fatty acids; sensitivity to phage T2; | fadL | −2.6 |
| regulator for gut (srl), glucitol operon; | srlR | −2.6 |
| methyl-galactoside transport and galactose taxis; | mglC | −2.6 |
| orf, hypothetical protein; | yhaF | −2.6 |
| putative formate acetyltransferase; | yfiD | −2.6 |
| orf, hypothetical protein; | yabI | −2.6 |
| putative thioredoxin-like protein; | ybbN | −2.6 |
| putative NAGC-like transcriptional regulator; | mlc | −2.6 |
| putative oxidoreductase; | yqhD | −2.6 |
| putative phosphatase; | yrfG | −2.6 |
| orf, hypothetical protein; | yhhZ | −2.6 |
| threonine dehydratase, catabolic; | tdcB | −2.7 |
| PTS system galactitol-specific enzyme IIC; | gatC | −2.7 |
| putative colanic acid polymerase; | wcaD | −2.7 |
| putative enzyme; | ybiX | −2.7 |
| ATP-binding component of ferric enterobactin transport; | fepC | −2.7 |
| putative sensory transduction regulator; | ybjN | −2.7 |
| sn-glycerol-3-phosphate dehydrogenase (anaerobic), large subunit; | glpA | −2.7 |
| deoxyguanosine triphosphate triphosphohydrolase; | dgt | −2.7 |
| PTS system, glucitol/sorbitol-specific IIC component, one of two; | srlA | −2.7 |
| galactoside permease (M protein); | lacY | −2.7 |
| orf, hypothetical protein; | ybjC | −2.7 |
| orf, hypothetical protein; | ycjF | −2.7 |
| putative oxidoreductase; | yeiQ | −2.7 |
| regulator of flagellar biosynthesis, acting on class 2 operons; | flhD | −2.7 |
| outer membrane receptor for ferric iron uptake; | fhuE | −2.7 |
| ATP-binding component of citrate-dependent iron(III) transport protein; | fecE | −2.7 |
| glutamate synthase, large subunit; | gltB | −2.7 |
| positive regulator for ctr capsule biosynthesis, positive transcription factor; | rcsA | −2.8 |
| cob(I)alamin adenolsyltransferase; | btuR | −2.8 |
| orf, hypothetical protein; | ycdQ | −2.8 |
| chaperone Hsp70; DNA biosynthesis; autoregulated heat shock proteins; | dnaK | −2.8 |
| putative colanic acid biosynthsis UDP-glucose lipid carrier transferase; | wcaJ | −2.8 |
| phosphatidylserine synthase; phospholipid synthesis; | pssA | −2.8 |
| putative lipoprotein; | ydhO | −2.8 |
| putative part of export apparatus for flagellar proteins; | flhB | −2.8 |
| orf, hypothetical protein; | b0919 | −2.8 |
| DNA-binding, ATP-dependent protease La; heat shock K-protein; | lon | −2.9 |
| nitrite reductase (NAD(P)H) subunit; | nirB | −2.9 |
| putative tagatose 6-phosphate kinase 2; | agaZ | −2.9 |
| cell division inhibitor, a membrane ATPase, activates minC; | minD | −2.9 |
| hydrogenase 4 membrane subunit; | hyfD | −2.9 |
| probable cytochrome Ni/Fe component of hydrogenase-2; | hybB | −2.9 |
| 1,4-dihydroxy-2-naphthoate --> dimethylmenaquinone; | menA | −2.9 |
| putative transport protein; | yqgA | −2.9 |
| putative lipase; | yiaL | −2.9 |
| sn-glycerol-3-phosphate dehydrogenase (anaerobic), large subunit; | glpA-r | −2.9 |
| putative transport; | yhgE | −2.9 |
| putative positive transcription regulator (sensor EvgS); | evgA | −2.9 |
| orf, hypothetical protein; | yieJ | −2.9 |
| synthesis of queuine in tRNA; | queA | −2.9 |
| galactitol-specific enzyme IIA of phosphotransferase system; | gatA | −3.0 |
| chaperone with DnaK; heat shock protein; | dnaJ | −3.0 |
| nitrite reductase activity; | nirC | −3.0 |
| regulator of ebg operon; | ebgR | −3.0 |
| orf, hypothetical protein; | b1044 | −3.0 |
| lipopolysaccharide core biosynthesis; phosphorylation of core heptose; | rfaP | −3.0 |
| phage lambda replication; host DNA synthesis; heat shock protein; | grpE | −3.0 |
| putative tRNA synthetase; | ybeK | −3.0 |
| pleiotrophic effects on 3 hydrogenase isozymes; | hypC | −3.0 |
| heat shock protein hslVU, proteasome-related peptidase subunit; | hslV | −3.0 |
| flagellar fliJ protein; | fliJ | −3.1 |
| putative hydantoin utilization protein; | ylbB | −3.1 |
| orf, hypothetical protein; | yibI | −3.1 |
| transcriptional represser of rpiB expression; | rpiR | −3.1 |
| orf, hypothetical protein; | b1995 | −3.1 |
| orf, hypothetical protein; | ykgF | −3.1 |
| orf, hypothetical protein; | b1903 | −3.1 |
| IS186 hypothetical protein; | yi81_1 | −3.2 |
| ATP-dependent serine activating enzyme; | entF | −3.2 |
| galactitol-1-phosphate dehydrogenase; | gatD | −3.2 |
| heat shock protein hslVU, ATPase subunit, homologous to chaperones; | hslU | −3.2 |
| ferredoxin-type protein: electron transfer; | napF | −3.2 |
| orf, hypothetical protein; | smp | −3.2 |
| formate dehydrogenase-N, nitrate-inducible, alpha subunit; | fdnG | −3.3 |
| putative transcriptional regulator LYSR-type; | yidZ | −3.3 |
| orf, hypothetical protein; | yihM | −3.3 |
| orf, hypothetical protein; | ygaH | −3.3 |
| nucleoside channel; receptor of phage T6 and colicin K; | tsx | −3.3 |
| orf, hypothetical protein; | yqcD | −3.3 |
| orf, hypothetical protein; | ycfA | −3.4 |
| orf, hypothetical protein; | ymfJ | −3.4 |
| anthranilate synthase component II; | trpD | −3.4 |
| guanine-nucleotide binding protein; | hypB | −3.4 |
| orf, hypothetical protein; | yjbF | −3.5 |
| orf, hypothetical protein; | napD | −3.5 |
| heat shock protein; | clpB | −3.6 |
| putative yhbH sigma 54 modulator; | yfiA | −3.6 |
| regulator for fec operon, periplasmic; | fecR | −3.6 |
| orf, hypothetical protein; | ylcC | −3.6 |
| GroEL, chaperone Hsp60, peptide-dependent ATPase, heat shock protein; | mopA | −3.6 |
| sn-glycerol-3-phosphate permease; | glpT | −3.6 |
| probable pilin chaperone similar to PapD; | ecpD | −3.7 |
| orf, hypothetical protein; | ybiU | −3.7 |
| periplasmic L-asparaginase II; | ansB | −3.8 |
| orf, hypothetical protein; | b1518 | −3.8 |
| putative 2-component regulator; | yieN | −3.8 |
| plays structural role in maturation of all 3 hydrogenases; | hypE | −3.9 |
| PTS system, enzyme 1, transcriptional regulator (with NPR and NTR proteins); | ptsP-r | −3.9 |
| polysaccharide metabolism; | yaiP | −3.9 |
| in glycine cleavage complex; | gcvH | −3.9 |
| orf, hypothetical protein; | yehT | −4.0 |
| putative peptidase; | b2324 | −4.0 |
| nitrate/nitrate sensor, histidine protein kinase acts on NarL regulator; | narX | −4.0 |
| putative PTS system enzyme IIA component, enzyme I; | b2383 | −4.1 |
| putative dehydrogenase subunit; | ykgE | −4.2 |
| IS30 transposase; | tra8_1 | −4.3 |
| orf, hypothetical protein; | ymdD | −4.3 |
| high-affinity transport of gluconate/gluconate permease; | gntT | −4.3 |
| outer membrane protein 1a (Ia; b; F); | ompF | −4.5 |
| histidinol-phosphate aminotransferase; | hisC | −4.5 |
| transcription-repair coupling factor; mutation frequency decline; | mfd-r | −4.6 |
| aminomethyltransferase of glycine cleavage system; | gcvT | −4.8 |
| acetate kinase; | ackA | −4.8 |
| orf, hypothetical protein; | b1593 | −5.0 |
| fumarate reductase, anaerobic, membrane anchor polypeptide; | frdC | −5.0 |
| putative fimbrial-like protein; | ycbQ | −5.0 |
| orf, hypothetical protein; | ybhP | −5.1 |
| anaerobically inducible L-threonine, L-serine permease; | tdcC | −5.1 |
| putative transcriptional regulator LYSR-type; | yafC | −5.1 |
| ferrous iron transport protein B; | feoB | −5.2 |
| orf, hypothetical protein; | ygcO | −5.7 |
| nitrite reductase (NAD(P)H) subunit; | nirD | −5.7 |
| aspartate ammonia-lyase (aspartase); | aspA | −5.7 |
| sn-glycerol-3-phosphate dehydrogenase (anaerobic), K-small subunit; | glpC | −5.9 |
| ferrous iron transport protein A; | feoA | −6.1 |
| transport of dicarboxylates; | dcuC | −6.2 |
| putative transport protein; | abrB | −6.2 |
| tagatose-bisphosphate aldolase 1; | gatY | −6.2 |
| heat shock protein; | ibpB | −6.5 |
| outer membrane porin protein; locus of qsr prophage; | nmpC | −6.6 |
| putative tagatose 6-phosphate kinase 1; | gatz | −6.8 |
| sn-glycerol-3-phosphate dehydrogenase (anaerobic); | glpB | −7.8 |
| ferredoxin-type protein: electron transfer; | napH | −7.9 |
| putative transcriptional regulator; | yihL | −8.0 |
| beta-D-galactosidase; | lacZ | −9.1 |
| GroES, 10 Kd chaperone binds to Hsp60 in pres. Mg-ATP; | mopB | −9.1 |
| putative resistance protein; | yhjX | −9.6 |
| periplasmic cytochrome c(552): plays a role in nitrite reduction; | nrfA | −12.7 |
| putative Rac prophage endopeptidase; | b1362 | −14.9 |

### Example 6

### Functional Grouping of the Responsive Gene

A number of the responding promoters/genes have been listed below and categorized according to physiological function or common regulation as described in the available literature.

TABLE 2

| Gene | b-number | Description | Function group | Fold |
|---|---|---|---|---|
| dksA | b0145 | dnaK suppressor protein | Acid shock | 4.1 |
| ybaS | b0485 | Putative glutaminase | Acid shock | 8.8 |
| nmpC | b0553 | Outer membrane protein, negatively regulated during | Acid shock | −6.6 |
| | | growth at low pH | | |
| dps | b0812 | DNA binding protein; global regulator, starvation | Acid shock | 4.4 |
| | | conditions | | |
| ycaC | b0897 | orf, hypothetical protein | Acid shock | 7.4 |
| gadC | b1492 | Putative glutamate: GABA antiporter | Acid shock | 24.9 |
| Asr | b1597 | Periplasmic acid shock protein | Acid shock | 19.2 |
| ydiZ | b1724 | ORF, hypothetical protein | Acid shock | 9.1 |
| yeaQ | b1795 | Putative transglycosylase-associated membrane | Acid shock | 6.9 |
| | | protein, acid inducible | | |
| yebV | b1836 | ORF, hypothetical protein | Acid shock | 6.9 |
| gabT | b2662 | GABA aminotransferase | Acid shock | 6.5 |
| gabP | b2663 | GABA permease | Acid shock | 8.2 |
| Slp | b3506 | Outer membrane protein induced after carbon | Acid shock | 3.6 |
| | | starvation | | |
| hdeB | b3509 | Periplasmic protein involved in acid resistance | Acid shock | 7.9 |
| hdeA | b3510 | Periplasmic protein involved in acid resistance | Acid shock | 7.7 |
| hdeD | b3511 | Integral membrane protein, unknown function, acid | Acid shock | 4.3 |
| | | inducible | | |
| gadE | b3512 | Activates glutamate-dependent acid resistance (yhiE) | Acid shock | 3.9 |
| gadW | b3515 | Transcriptional regulator, AraC-type (yhiW) | Acid shock | 3.0 |
| yiaG | b3555 | Putative transcription factor, acid inducible | Acid shock | 10.1 |
| bolA | b0435 | Morphogene, induced by osmotic shock | Osmotic shock | 13 |
| dacC | b0839 | D-alanyl-D-alanine carboxypeptidase; regulated by | Osmotic shock | 3.6 |
| | | bolA | | |
| osmC | b1482 | Osmotically induced protein | Osmotic shock | 7.2 |
| katE | b1732 | Catalase, hydroperoxidase HPII(III) | Osmotic shock | 9.9 |
| osmE | b1739 | Osmotically inducible, activator of ntrL | Osmotic shock | 11.7 |
| otsA | b1896 | Trehalose-6-phosphate synthase | Osmotic shock | 5.2 |
| otsB | b1897 | Trehalose-6-phosphate phosphatase | Osmotic shock | 5.6 |
| yehX | b2129 | Part of putative osmoprotectant system, yehZYXW | Osmotic shock | 5.6 |
| yehY | b2130 | Part of putative osmoprotectant system, yehZYXW | Osmotic shock | 4.2 |
| yehZ | b2131 | Part of putative osmoprotectant system, yehZYXW | Osmotic shock | 7.1 |
| proW | b2678 | High affinity transport system for glycine, betaine and | Osmotic shock | 7.9 |
| | | proline | | |
| osmY | b4376 | Hyperosmotically inducible periplasmic protein | Osmotic shock | 6.4 |
| appC | b0978 | Cytochrome bd terminal oxidase, subunit I | Redox | 3.5 |
| appB | b0979 | Cytochrome bd terminal oxidase, subunit II | Redox | 47.2 |
| ndh | b1109 | Transfer of electrons from NADH to the respiratory | Redox | 3.1 |
| | | chain | | |
| aer | b3072 | Aerotaxis sensor receptor | Redox | 14.3 |
| pspA | b1304 | Phage shock protein | Phage shock | 6.7 |
| pspB | b1305 | Phage shock protein | Phage shock | 6.9 |
| pspC | b1306 | Phage shock protein | Phage shock | 15.8 |
| pspD | b1307 | Phage shock protein | Phage shock | 13.5 |
| pspE | b1308 | Phage shock protein | Phage shock | 9.4 |
| dnaK | b0014 | Heat shock protein, chaperone | Heat shock | −2.8 |
| dnaJ | b0015 | Heat shock protein, chaperone | Heat shock | −3.0 |
| lon | b0439 | Heat shock protein, protease | Heat shock | −2.9 |
| clpB | b2592 | Heat shock protein, chaperone | Heat shock | −3.6 |
| grpE | b2614 | Heat shock protein, stimulates DnaK ATPase | Heat shock | −3.0 |
| ibpB | b3686 | Heat shock protein, chaperone | Heat shock | −6.5 |
| hslU | b3931 | Heat shock protein, protease(?) | Heat shock | −3.2 |
| hslV | b3932 | Heat shock protein | Heat shock | −3.0 |
| mopB | b4142 | Heat shock protein, GroES, chaperone | Heat shock | −9.1 |
| mopA | b4143 | Heat shock protein, GroEL, chaperone | Heat shock | −3.6 |
| sfmC | b0531 | Periplasmic pilus chaperone | Protein folding | 11.4 |
| crcA | b0622 | phoP/phoQ-dependent enzyme, lipid A modifying | AMP resistance | 11.7 |
| | | enzyme | | |
| ais | b2252 | Homolog to polymyxin resistance gene pmrG in | AMP resistance | 14.1 |
| | | S. typhimurium | | |
| b2253 | b2253 | Putative aminotransferase; modification of lipid A with | AMP resistance | 8.4 |
| | | aminoarabinose | | |
| b2254 | b2254 | Putative glycosyl transferase; LPS biosynthesis (pmrF | AMP resistance | 5.5 |
| | | homolog) | | |
| b2256 | b2256 | Homolog to polymyxin resistance gene in | AMP resistance | 4.0 |
| | | S. typhimurium | | |
| phoQ | b1129 | Part of two-component regulatory system with phoP | Mg²⁺-stimulon | 3.7 |
| phoP | b1130 | Sensor In two-component regulatory system with | Mg²⁺-stimulon | 2.8 |
| | | phoQ | | |
| mgtA | b4242 | Magnesium transport, PhoP/PhoQ-dependent | Mg²⁺-stimulon | 7.5 |
| mgrB | b1826 | Hypothetical protein; induced by low magnesium | Mg²⁺-stimulon | 4.6 |
| hemL | b0154 | Glutamate-1-semialdehyde aminotransferase | Mg²⁺-stimulon | 8.3 |
| rstB | b1609 | Putative sensor kinase | Mg²⁺-stimulon | 3.6 |
| nagA | b0677 | N-acetylglucosamine-6-phosphate deacetylase | Mg²⁺-stimulon | 3.7 |
| nagB | b0678 | glucosamine-6-phosphate deaminase | Mg²⁺-stlmulon(?) | 8.8 |
| narX | b1222 | nitrate/nitrate sensor, histidine protein kinase | Nitrogen assimilation | −4.0 |
| narY | b1467 | Cryptic nitrate reductase 2, beta subunit | Nitrogen assimilation | 11.0 |
| narZ | b1468 | Crytic nitrate reductase 2, alpha subunit | Nitrogen assimilation | 4.9 |
| narU | b1469 | Nitrite extrusion protein 2 | Nitrogen assimilation | 15.1 |
| nirB | b3365 | Nitrite reductase, NAD(P)H, large subunit | Nitrogen assimilation | −2.9 |
| nirD | b3366 | nitrite reductase (NAD(P)H) subunit | Nitrogen assimilation | −5.7 |
| nirC | b3367 | Potential nitrite transporter | Nitrogen assimilation | −3.0 |
| nrfA | b4070 | Component of nitrite reductase complex | Nitrogen assimilation | −12.7 |
| sfsA | b0146 | Probable regulator for maltose metabolism | Carbon metabolism | 6.4 |
| sfcA | b1479 | Putative NAD-linked malic enzyme | Carbon metabolism | 5.8 |
| amyA | b1927 | Cytoplasmic alpha-amylase | Carbon metabolism | 5.8 |
| b2097 | b2097 | Class I Fructose 1,6 bisphosphate aldolase | Carbon metabolism | 8.1 |
| glk | b2388 | Glucokinase | Carbon metabolism | 3.4 |
| talA | b2464 | Transaldolase A | Carbon metabolism | 11.7 |
| tktB | b2465 | Transketolase 2 isoenzyme | Carbon metabolism | 26.5 |

1. A method for identifying DNA control sequences, wherein the transcriptional profiles of said control sequences are regulated by the presence of an antimicrobial polypeptide, comprising the steps:
a) providing a nucleotide sequence encoding the antimicrobial polypeptide, and expressing the said antimicrobial polypeptide in a host cell;
b) isolating mRNA from the host cell in step (a); and
c) obtaining a transcription profile by analyzing the mRNA from step (b).

2. The method according to claim 1, wherein the transcription profile is obtained by a DNA array analysis of the isolated mRNA.

3. The method according to claim 2, wherein mRNA from step (b) is labeled and hybridized to the DNA-array.

4. The method according to claim 2, wherein cDNA, provided from the mRNA of step (b), is labeled and hybridized to the DNA-array.

5. The method according to claim 1, wherein the host cell comprises a bacterial cell.

6. The method according to claim 5, wherein the bacteria cell comprises pathogenic bacteria.

7. The method according to claim 5, wherein the bacteria comprises E. coli, Bacilli, Lactococci, Lactobacilli, Streptococci, Staphylococci, Aerococci, Enterobacter, Enterococci, Pseudomonas sp., Acinetobacter, Bacteriodes, Bordetella, Citrobacter, Klebsiella, Micrococci, Morganella, Mycobacteria or Stenotrophomonas.

8. The method according to claim 1, wherein the antimicrobial polypeptide is chosen from the group consisting of alpha helical peptides.

9. The method according to claim 8, wherein the alpha helical peptide is novispirin.

10. A host cell comprising a nucleotide sequence encoding a novel antimicrobial polypeptide or an antimicrobial polypeptide variant and a control sequence identifiable by the method of claim 1, wherein the control sequence is operably linked to a reporter gene.

11. The host cell according to claim 10, wherein a reporter protein encoded by the reporter gene comprises beta-galactosidase, luciferase, GFP, RFP, BFP, YFP, and drug resistance markers.

12. The host cell according to claim 10, wherein the control sequence comprises promoters chosen from the subgroups comprising promoters controlling genes involved in acid shock, osmotic shock, membrane biogenesis and repair, redox, phage shock, protein folding, AMP resistance, cell wall, nitrate assimilation, carbon metabolism or heat shock.

13. The host cell according to claim 12, wherein the promoters control genes involved in acid shock, osmotic shock or phage shock.

14. The host cell according to claim 13, wherein the genes comprises Asr, gadB, gadC, KatE, OsmE, ProW and pspABCDE.

15. The host cell according to claim 9, wherein the antimicrobial polypeptide comprises alpha helical polypeptides or a variant of said polypeptide.

16. A method of screening for novel antimicrobial polypeptides and antimicrobial polypeptide variants, comprising the steps:
a) expressing a library of potential antimicrobial polypeptides or antimicrobial polypeptide variants in a host cell according to claim 10;
b) selecting cells on the basis of the reporter gene expression level.

17. The method according to claim 16, further comprising the step:
c) isolating and identifying the novel polypeptide or the polypeptide variants from the host cells having up- or down-regulated reporter gene expression levels compared to an identical cell expressing the no antimicrobial polypeptide or parent antimicrobial polypeptide.

18-23. (canceled)
